(19) United States
(12) Patent Application Publication
(10) Pub. No.: US 2008/0132991 A1
(43) Pub. Date: Jun. 5, 2008

(54) Method for Ionically Cross-Linking Gellan Gum for Thin Film Applications and Medical Devices Produced Therefrom

(76) Inventors: Leonard Pinchuk, Miami, FL (US); Yasushi Pedro Kato, Pembroke Pines, FL (US); Marc Ramer, Weston, FL (US)

Correspondence Address: GORDON & JACOBSON, P.C., 60 LONG RIDGE ROAD, SUITE 407, STAMFORD, CT, 06902, US

(21) Appl. No.: 11/565,065

(22) Filed: Nov. 30, 2006

(51) Int. Cl. A61F 2/82; B05D 7/00; B05D 3/10

(52) U.S. Cl. 623/1.13; 427/2.24; 427/2.25; 427/337

(57) ABSTRACT

A method for producing ionically cross-linked gellan gum includes dissolving the material in a first liquid solution that includes a dissolving liquid. The first liquid solution is applied to a workpiece to form a polysaccharide-based coating on the workpiece. The coating is dried to remove a substantial portion of the dissolving liquid. Subsequent to drying, the coating is exposed to a second liquid solution that includes a compound that promotes ionic cross-linking of the coating. In the preferred embodiment, the dissolving liquid comprises water and possibly a polar solvent. The ionic cross-linking compound preferably comprises a divalent cation such as calcium (Ca²⁺), or possibly strontium (Sr²⁺), magnesium (Mg²⁺), barium (Ba²⁺), or other multivalent ions. Such a method forms a uniform ionically cross-linked film and/or coating for diverse applications, including medical devices such as implantable vascular grafts, stent-grafts and/or stents.

### BACKGROUND OF THE INVENTION,

1. Field of the Invention

This invention relates to methods for cross-linking gellan gum and products produced from these materials.

2. State of the Art

Gellan gum is a hydrocolloid polysaccharide produced by the microorganism Sphingomonas elodea. It is manufactured from the fermentation of a readily available carbohydrate raw material. As needed, deacylation is conducted with alkali. Molecular weights range from 1-2,000,000 Daltons. The naturally occurring high-acyl form is thermo-reversible from elevated temperatures (70-80° C.) while the low acyl form is not.

The molecular structure of gellan gum is a straight chain based on repeating units of glucose, rhamnose, and glucaronic acid. The acyl groups in the natural (acylated) form include acetate and glycerate. Both substituents reside on the glucose residue and average one glycerate per repeat and one acetate every other repeat. The acylated form produces soft, elastic, non-brittle gels. The deacylated form is completely devoid of acyl groups. It produces firm, non-elastic, brittle gels.

Gellan gum is available as a free-flowing white powder. Typically gellan gum is dissolved in water and mixed to produce a 0.03-1% solids content solution. The viscosity of the solution increases with solids content and graduates from a “fluid gel” to a semisolid at approximately 0.2% (w/w). The dissolution process is aided by low temperatures and low (approximately <0.03%) ion content, since higher temperatures encourage clumping and modest ion content increases the powder's hydration temperature. Gellan gums are generally not soluble in polar solvents such as alcohol. Chemicals such as glycerin may be used as a processing aid to encourage powder dispersion.

Gellan gums tend to remain liquid at elevated temperatures (above approximately 70° C.) and gel when brought below this temperature. Gellan gum demonstrates the characteristic of “snap-setting,” meaning it gels very quickly when the setting temperature is reached. This gel is strengthened with addition of a cross-linking agent, such as monovalent, divalent, or multivalent ions in low concentrations (approximately 0.05-0.15%). Higher salt concentrations will cross-link the gel and precipitate unbonded salt solids.

Gellan gum has been used for in-situ scleral applications as described in Viegas et al. (U.S. Pat. No. 6,136,334). However, Viegas et al. describes pH buffered gels placed for the purpose of facilitating drug delivery to the eye. Other literature references include: Alupei, I. C.; Grecu, I.; Gurlui, S.; Popa, M.; Strat, G.; Strat, M., “The study of the structure and optical properties of gellan-PVA, gellan-PVP and gellan-PVI composites,” Proc. Int. Symp. Electrets. 2002, pp. 426-429; and Balasubramaniam, J.; Kumar, M. T.; Pandit, J. K.; Kant, S., “Gellan-based scleral implants of indomethacin: In vitro and in vivo evaluation,” Drug Delivery: Journal of Delivery and Targeting of Therapeutic Agents, 11/6 (371-379), 2004.

Gellan gum has also been used in devices for insulin delivery as described Epstein et al. (U.S. Pat. No. 6,923,996). However, Epstein et al. describes a genus of polymers that includes gellan gum for medical implants without highlighting the special benefits of the gum. See also Li, J; Kamath, K; Dwivedi, C, “Gellan film as an implant for insulin delivery,” J. Biomater. Appl., 15(4), April 2001, pp. 321-43.

Polymer-based coatings have been proposed for medical devices including implantable, percutaneous, transcutaneous, or surface applied medical devices, such as vascular stents, stent-grafts, grafts, catheters, bone screws, joint repair implants, tissue repair implants, feed tubes, shunts, endotracheal tubes, etc. See U.S. Patent Pub. 2003/0158958, U.S. Patent Pub. 2003/004559 and U.S. Pat. No. 6,723,350. A stent is a generally longitudinal tubular device formed of biocompatible material, preferably a metallic or plastic material. Stents are useful in the treatment of stenosis and strictures in body vessels, such as blood vessels. It is well known to employ a stent for the treatment of diseases of various body vessels. The device is implanted either as a “permanent stent” within the vessel to reinforce collapsing, partially occluded, weakened, or abnormally dilated sections of the vessel or as a “temporary stent” for providing therapeutic treatment to the diseased vessel. Stents are typically employed after angioplasty of a blood vessel to prevent restenosis of the diseased vessel. Stents may be useful in other body vessels, such as the urinary tract and the bile duct. A stent-graft employs a stent inside or outside a graft. The graft is generally a longitudinal tubular device formed of biocompatible material, typically a woven polymeric material such as Dacron or polytetrafluroethylene (PTFE). Stent-grafts and vascular grafts are typically used to treat aneurysms in the vascular system. Bifurcated stent-grafts and bifurcated vascular grafts can be used to treat abdominal aortic aneurysms. It is desirable that grafts are impermeable to body fluid (e.g., blood) that flows through the graft such that the body fluid does not leak out through its wall(s).

Stents and stent-grafts typically have a flexible configuration that allows these devices to be configured in a radially compressed state for intraluminal catheter insertion into an appropriate site. Once properly positioned, the devices radially expand such that they are supported within the body vessel. Radial expansion of these devices may be accomplished by an inflatable balloon attached to a catheter, or these devices may be of the self-expanding type that will radially expand once deployed.

U.S. Patent Pub. No. 2003/0158598 to Ashton et al. describes the coating of stents, stent-grafts, and grafts with a drug-loaded polymer matrix and a polysaccharide (pectin). The pectin degrades over time and is used to control the release rate of the drug loaded into the polymer matrix. U.S. Patent Pub. No. 2003/0004559 describes a vascular graft employing inner and outer microporous expanded polytetrafluoroethylene (ePTFE) tubes that are formed in separate extrusion processes. An intermediate elastomeric layer is disposed between the two tubes. The intermediate layer may be impregnated with a polysaccharide gel to provide enhanced sealing capabilities.

U.S. Pat. No. 6,723,350 to Burrell et al. describes a lubricious coating applied to a wide variety of medical devices. The coating can be realized from polysaccharide-based compound prepared from a liquid medium having a gel-like consistency.

Typically, a polysaccharide solution remains in solution form until a gelling agent is introduced. For pectin, calcium (Ca²⁺) ions are added to the solution for gelling. These ions require a minimum concentration in order to yield gels with desired properties. Excessive concentrations cause pre-gelation and a tendency for syneresis to occur. Syneresis is the process of moisture expulsion (or removal) as the gel shrinks or conformation changes.

In each of these applications, the methodology for applying pectin-based film to the respective device impedes or complicates the formation of a uniform coating. Furthermore, the polysaccharide-based films of the prior art are often of more limited flexibility and less pliable. This characteristic can hinder the durability of these coatings making them less acceptable in the medical applications market.

Thus, there remains a need in the art to provide an improved method for the formation of biocompatible films and impregnates as well as coatings that are suitable for medical device applications such as vascular stents, stent-grafts, and vascular grafts requiring uniform coatings and pliability.

### SUMMARY OF THE INVENTION

It is therefore an object of the invention to provide a method for preparing a biocompatible film that has improved flexibility for body implants to assist physicians in maneuverability and in atraumatic attachment to the vasculature.

It is another object of the invention to provide a method for preparing a biocompatible film that has a pliable handle that is suitable for applications requiring coatings for body implants.

It is still another object of the invention to provide a method for preparing a biocompatible film that has a color that can assist physicians in recognition of implanted medical devices coated with the film.

It is a further object of the invention to provide a method for coating medical devices, such as a vascular graft, stent, or stent-graft, that has a uniform coating of ionically cross-linked gellan gum.

It is a further object of the invention to provide a method for preparing such coatings with a gradient such that the outer surface of the material has a higher cross-linking density than the inner surface of the material.

In accordance with these objects, a biocompatible gellan gum based film is provided that has improved qualities of flexibility and color. The film is suitable for application to a wide variety of implantable medical devices such as stents, stent-grafts, and vascular grafts.

According to a first, preferred embodiment, a method is provided for producing an ionically cross-linked gellan gum based film for coating medical implants where gellan gum is first dissolved in solution and applied to the medical implant. The applied solution is then dried to substantially remove the dissolving liquid. This process produces a gellan gum coating. The gellan gum coating is then ionically cross-linked by the addition of a solution that contains a multivalent cation. The resulting coating is substantially white and flexible.

According to a second embodiment, the method of the first embodiment is used to produce a film having a density gradient across the film's thickness. This density gradient is produced by carefully exposing the dried coating to the cross-linking agent in a more controlled manner so that the inner and outer cross-linking densities vary across the body of the film.

In another embodiment of the invention, a method for manufacturing an implantable medical device is described by applying a solution of gellan gum to the desired device. The solution is then dried and a second solution containing cross-linking agents is used to cross-link the gellan gum on the surface of the device.

According to another embodiment, calcium chloride is used in solution to initiate cross-linking of a dried gellan gum solution.

Additional objects and advantages of the invention will become apparent to those skilled in the art upon reference to the detailed description taken in conjunction with the provided figure.

### BRIEF DESCRIPTION OF THE DRAWINGS

FIG. 1 is a schematic diagram of a vascular graft formed with an ionically cross-linked gellan gum coating in accordance with the present invention.

### DETAILED DESCRIPTION OF THE PREFFERRED EMBODIMENTS

For the purposes of this patent application, “ionic cross-linking” refers to a process wherein a polymer (e.g., gellan gum) is transformed by the formation of ionic bonds between chains of the polymer. The ionic bonds require multivalent counter-ions that form bridges between polymeric chains. A polymer is “ionically cross-linked” after it has been subjected to such ionic cross-linking. A thin film is a layer of material that is no larger than 1 millimeter (mm) in thickness.

In accordance with the objects of this invention, an improved method is provided for producing an ionically cross-linked gellan gum in a first embodiment of this invention. Gellan gum is dispersed in a gellan gum-dispersing liquid. This dispersion is then added to a gellan gum-dissolving liquid. The resulting liquid solution is then exposed to another liquid solution that includes a compound that induces ionic cross-linking of the gellan gum-based liquid solution. The cross-linked gellan gum-based solution is then applied to a workpiece to form a gellan gum-based coating on the workpiece or a gellan-based impregnate in the interstices of the workpiece. Excess gel on the workpiece is removed. The gellan gum-coated workpiece is then exposed to another solution to remove pyrogens and excess cross-linking agents. The workpiece is then exposed to another solution to plasticize the workpiece. Finally, the gellan gum-based coating is dried to remove a substantial portion of the gellan gum-dissolving liquid.

It will be appreciated that this methodology forms a uniform, ionically cross-linked gellan gum film and/or coating suitable for diverse applications, including medical devices such as implantable vascular grafts, stents, etc.

Unlike films produced from other polysaccharide materials, gellan gum offers the unique combination of yielding bright white thin films that are also more flexible as compared to films produced by other polysaccharides. Gellan gum also produces films that have the added benefit of being less brittle than films produced from other polysaccharides. Consequently, the combination of these qualities offers special benefits in applications of medical devices and implant films. As an example, physicians tend to be hesitant to accept medical devices that are off-white or yellowish in color. Individuals generally associate discolored devices as being old or unclean and therefore prefer devices that are pristine white in appearance. Conventional vascular grafts are coated with gels made from collagen or gelatin and are often-times yellow in appearance. Further, one batch of collagen or gelatin can be slightly yellower than others and physicians may be discriminatory in these differences and often times return these off-colored devices to the vendor. Gellan-based film with its pristine white color would generally be more acceptable to a physician.

In addition to the color, films generated by gellan gum are soft and supple when compared to films from other polysaccharides or from gelatin and collagen. The suppleness is important for two reasons, first the graft is easier to maneuver under the skin (when tunneled into place) and to follow the contour of the body when implanted. Second, the softness of the graft is important in that it is desirable not to place undue stresses on the native artery when sutured in place. Stiff grafts may pull on the anastomosis and cause disruptions or undue scarring of the tissue.

Gellan gum also has a significant, added benefit in that it does not carry prions for Mad Cow disease, unlike collagen.

According to one embodiment, the gellan gum-dissolving liquid comprises water or possibly a polar solvent. The ionic cross-linking compound preferably comprises a divalent cation such as calcium (Ca²⁺), barium (Ba²⁺), magnesium (Mg²⁺), strontium (Sr²⁺), and/or other multivalent ions.

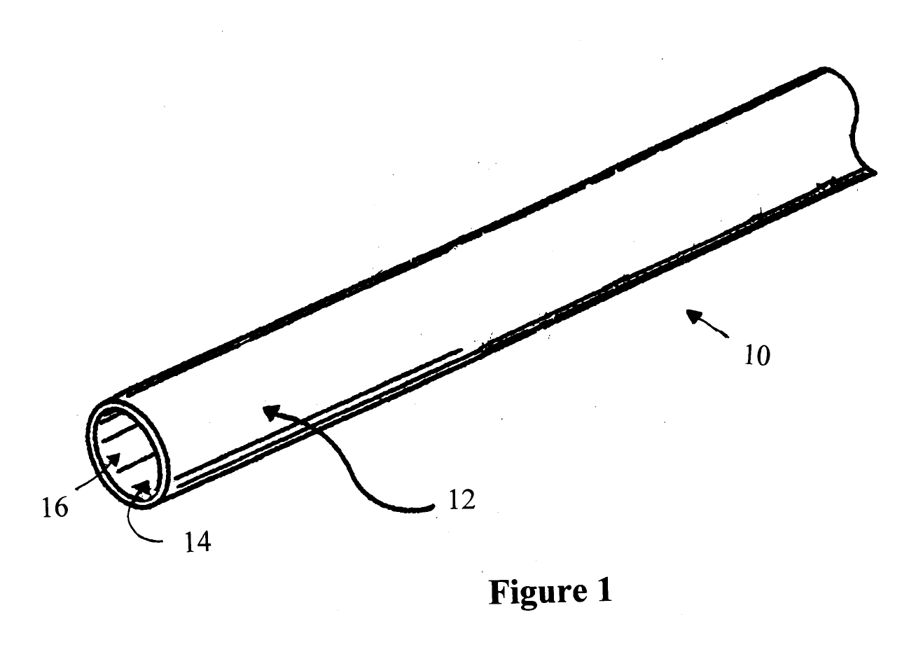

FIG. 1 is a schematic diagram of a vascular graft formed with an ionically cross-linked polysaccharide-based coating in accordance with the present invention.

In accordance with the present invention, a film or coating of ionically cross-linked gellan gum is realized as follows.

First, a gellan gum powder is dissolved in water to produce a homogenous solution of gellan gum. This dissolving processing can be aided by using cold water. Monovalent or divalent ions may be added to the solution in low concentration to aid in dispersing the gellan gum therein. The concentration of gellan gum in the solution can vary between 0.025% to approximately 1% as desired. The gellan gum solution is coated, sprayed, or impregnated onto a workpiece and dried to remove water and any solvents, which produces a dried film of gellan gum on the workpiece. Preferably, such application of the solution produces a thin film. The drying process can be accomplished by subjecting the gellan gum-coated workpiece to ambient temperatures or to elevated temperatures in a warm oven. Thicker films or coatings of gellan gum can be produced by applying/drying additional gellan gum layers on top of the base layer or by using a higher solids content gellan gum solution. The dried film of gellan gum may have some retained solvents (for example, between 0 to 20% of the water and solvents may be left behind in the film). The dried film of gellan gum may be removed from the workpiece, if desired.

A liquid solution of calcium chloride in water is prepared. The concentration of calcium chloride can range from near zero to 2% (weight/weight) and preferably between 0.05-0.5% (weight/weight) and most preferably between 0.05% and 0.15% (weight/weight). Other compound(s) can be mixed into the liquid calcium chloride solution as long as the other compound(s) do not compete or steal the calcium ions that are present in the liquid calcium chloride solution. The dried gellan gum film (and possibly the workpiece if the film was not removed therefrom) is exposed to the liquid calcium chloride solution at a predetermined temperature (e.g., room temperature) for a predetermined time (e.g., 30 minutes). The calcium divalent cations (Ca²⁺ ions) of the liquid solution form bridges between polymeric chains of the gellan gum film submersed therein to thereby ionically cross-link the gellan gum. The calcium chloride concentration as well as the temperature and time of the exposure to the calcium chloride will affect the degree of the ionic cross-linking up to a point of saturation. Therefore, different degrees of ionic cross-linking can be achieved by varying the calcium chloride concentration as well as the temperature and time of exposure to the calcium chloride solution. These different degrees of ionic cross-linking can provide for different gellan gum properties as desired. Moreover, as the gellan gum can be built up to a desired thickness by multiple coatings, the calcium chloride concentration and the exposure time can be controlled to produce a gradient of ionically cross-linked layers that have a higher ionically cross-linked density on the outside compared to the inside (inner) layer(s).

The calcium reactivity of a specific gellan gum depends upon its degree of esterification and the uniformity among molecules of the lot. When Ca²⁺ ions are added to the gum solution for gelling, the solution starts to gel and thicken. Gellan differs from pectin in that it gels when it is cooled below 70° C. Above 70° C. it remains fluidic even in the presence of monovalent and divalent ions such as Ca²⁺.

One skilled in the art will realize that the ionic cross-linking agent of the bath can comprise other divalent cations such as calcium (Ca²⁺), barium (Ba²⁺), magnesium (Mg²⁺), strontium (Sr²⁺), and/or other multivalent ions.

In yet other embodiments of this invention, a uniform ionically cross-linked gellan gum coating can be applied to other medical devices, such as implantable stents, stent-grafts, vascular grafts, and other implantable medical devices. In these applications, a gellan gum is coated, sprayed, or impregnated onto the respective device and dried to remove water and any solvents, which produces a dried film of gellan gum on the device. Preferably, such application of gellan gum produces a thin film. However, the gellan gum material can be built up to a desired thickness by multiple coatings/drying steps or by using a higher solids content gellan gum solution as described above. The gellan gum-coated device is then immersed (or otherwise subjected) to a bath of 5% calcium chloride (or other suitable ionic cross-linking agent as described above) in order to ionically cross-link the gellan gum coating. The gellan gum-coated device is then preferably rinsed, immersed in distilled water, and immersed in glycerin in order to plasticize the gellan gum coating. Finally, the gellan gum-coated device is dried. The uniform ionically cross-linked gellan gum coating can be used to render surfaces of the device impermeable to bodily fluid (e.g., blood in vascular applications) or possibly for controlling the release rate of therapeutic drugs loaded into a release structure (e.g., polymer matrix) disposed under the gellan gum coating.

The gellan gum coatings/films described herein can also be used as a lubricious coating layer for a wide variety of medical devices, including catheters, bone screws, joint repair implants, tissue repair implants, feed tubes, shunts, endotracheal tubes, etc. The gellan gum coatings/films can also be applied to a medical device and used to hold a therapeutic drug for drug delivery purposes. The drug can be mixed with the liquid gellan gum solution and subsequently applied to part of the medical device, where it is dried and then subjected to a cross-linking agent(s). The drug must not react with the gellan gum nor with the cross-linking agent(s) to form other entities. In this application, the drug can be eluted from the gellan gum coating/film as the gellan gum coating/film slowly degrades over time.

In accordance with the present invention, an ionically cross-linked gellan gum coating was applied to a tubular structure 12 of a graft 10. The gellan gum coating renders the tubular structure 12 impermeable to blood flowing through a central lumen 14. Central lumen 14 is defined by an inner wall surface 16 of the tubular structure 12.

The gellan gum coating will degrade with time in the body by the action of inflammatory cells and host tissue will take its course of healing from inflammation, proliferative to remodeling phases. In the inflammatory phase (which usually takes a few days), platelet aggregation and thrombin will coat the surface and macrophages will start to degrade the gellan gum coating by phagocytosis and possibly enzymatic and oxidative degradation. In the proliferative phase and the final remodeling phase (which usually lasts a few days to a few weeks/months), extracellular matrix and collagen will be formed by fibroblasts onto the interstices of the tubular structure, thereby providing a replacement blood-impermeable layer as a substitute for the gellan gum layer. The ionically cross-linked gellan gum coating may be applied to the tubular structure 12 in analogous methods described below.

### EXAMPLE 1

First, gellan gum powder is mixed with glycerin to produce a slurry of well-distributed (non-clumped) gellan gum powder. The slurry in then added in small increments to a vigorously stirring solution containing a low concentration of Ca²⁺ ions (approximately 0.03%) and cold water. The solution is then gradually heated to 85° C. while stirring vigorously at both the bottom and surface of the solution. The solution may use gellan gum concentrations ranging from 0.03% to 1% as desired. The gellan gum solution is coated or impregnated into a workpiece, the excess gel is removed, the workpiece is soaked in water, then soaked in a glycerin solution, and finally the workpiece is dried to remove water, which produces a uniform coating of gellan gum on the workpiece. Such drying can be accomplished by subjecting the gellan gum-coated workpiece to ambient temperatures or to elevated temperatures in a warm oven. Thicker coatings of gellan gum can be produced by applying/drying additional gellan gum layers on top of the base layer or by using a higher solids content gellan gum solution. The dried coating of gellan gum can have some (for example, 0-20%) of the water and solvents left in the coating. The dried coating of gellan gum may be removed from the workpiece, if desired.

In the example above, a liquid solution of calcium chloride in water is prepared. The concentration of calcium chloride can range from near zero to 10% (weight/weight) and preferably between 0.05-5% (weight/weight) and most preferably between 0.05-0.15% (weight/weight). Other compounds can be mixed into the liquid calcium chloride solution as long as the other compound(s) do not compete or steal the calcium ions that are present in the liquid calcium chloride solution. The gellan gum solution (and possibly the workpiece if the coating was not removed therefrom) is exposed to the liquid calcium chloride solution at a predetermined temperature (e.g., 85° C.) for a predetermined time (e.g., 2 minutes). The calcium divalent cations (Ca²⁺ ions) of the liquid solution form bridges between the polymeric chains of the gellan gum submersed therein to thereby ionically cross-link the gellan gum. The calcium chloride concentration as well as the temperature and time of the exposure to the calcium chloride will affect the degree of ionic cross-linking up to a point of saturation. Therefore, different degrees of ionic cross-linking can provide for different gellan gum properties as desired. Moreover, as the gellan gum can be built up to a desired thickness by multiple coatings, the calcium chloride concentration and the exposure time can be controlled to product a gradient of ionically cross-linked density on the outside compared to the inside (inner) layer(s).

### EXAMPLE 2

Gellan gum solutions were made by dissolving 0.5 g high acyl gellan gum and 10 g glycerin in 79.5 g of distilled water. The solution was heated to 85° C. and 10 g of either 1.5% BaCl₂ or 1.5% CaCl₂ was added. This resulted in a solution with 0.5% gellan gum, 0.15% BaCl₂ (or CaCl₂) and 10% glycerin (all % are weight/weight). Ten milliliters of the solution was placed in a weighing dish and allowed to dry at ambient temperature, then 50° C. overnight. Other solutions were made without the cross-linker addition to solution; rather, the cross-linker was added as a 5% solution to the surface of the room-temperature gels. Segments of unsealed, woven double velour vascular graft were also dipped in the solution, squeezed to remove excess gel, and dried overnight at 50° C. The films were sterilized either by e-beam or ethylene oxide (EtO) gas. A #5 punch was used to make gel disks from the films, and the disks were submerged in 10 milliliter phosphate buffered saline with 5% isopropanol. The immersed disks were incubated at 37° C. for 1 to 14 days, during which they were evaluated qualitatively for swelling/dissolution and quantitatively for weight loss (vs. pre-soak weight). In addition, the graft segments were tested for permeability and suture retention. The data suggested: (1) the gellan gum coating is not sterilizable by e-beam; (2) adding CaCl₂ to gel solution to achieve 0.15% yields the best coating, as shown by having the least dissolution and moderate swelling; and (3) coated grafts in the preferred configuration showed low permeability and high suture retention strength.

### EXAMPLE 3

Gellan gum solutions were made to include 0.5% high acyl gellan gum and cross-linker concentrations of 0.125%, 0.25%, 0.5%, 1%, and 2%. Film disks and grafts were made from each solution. The disks and grafts were soaked in phosphate buffered saline at 37° C. for 1, 2, 4, and 7 days. The disks at each timepoint were assessed for weight and thickness change while the grafts were tested for permeability. In addition, film disks were also created from a collagen slurry, and collagen-coated grafts were treated and tested identically to the gellan gum-coated grafts. The data showed that the baseline (0 day) grafts created using 2% cross-linker concentration had very high permeability, and those made with cross-linker concentrations of 0.5%, 1%, and 2% had high levels of precipitate in the solution after 1 day. Disks containing 0.25% cross-linker content also showed precipitation in solution at 2 days. Disk weight gain and thickness increase was much greater for gellan gum disks than for collagen. Graft permeability for units containing 0.125% or 0.25% cross-linker reached or approached 0 cc/cm²/min at all timepoints.

### EXAMPLE 4

Gellan gum coated, woven double velour vascular grafts were assembled with 0.5% high acyl gellan gum, 0.15% CaCl₂, 10% glycerin, and distilled water. After EtO sterilization, they were tested using a variety of performance tests in common practice. The data suggest that the grafts perform well in kink resistance, permeability (acutely and after saline soaking), suture retention strength, longitudinal tensile strength, and coating uniformity.

### EXAMPLE 5

Additional gellan gum coated, woven double velour vascular grafts were assembled with 0.5% high acyl gellan gum, 0.15% CaCl₂, 10% glycerin, and distilled water. Some were EtO sterilized. They were assessed for weight gain (vs. pre-coating) and permeability (pre- vs. post-sterilization). The data showed that coated graft weight gain is well controlled, permeability is less than 2 cc/cm²/min pre-sterile and higher post-sterile.

### EXAMPLE 6

Additional gellan gum coated, woven double velour vascular grafts were assembled with 1% high acyl gellan gum, 0.15% CaCl₂, and distilled water. Glycerin was added to solution at 3.5% as a powder dispersion aid and at 10%, after dipping, as a plasticizing agent. Units were EtO sterilized. The data showed that weight gain was well controlled and permeabilities (pre- and post-sterile) were usually ≦1 cc/cm²/min.

There have been described and illustrated herein several embodiments of a method for forming a uniform ionically cross-linked gellan gum film or coating and products based thereon. While particular embodiments of the invention have been described, it is not intended that the invention be limited thereto, as it is intended that the invention be as broad in scope as the art will allow and that the specification be read likewise. Thus, while particular concentrations, temperatures, and heating times have been disclosed, it will be appreciated that other such parameters can be used as well. In addition, while applications for particular types of implantable medical devices have been disclosed, it will be understood that the principles of the present invention can be used for other implantable medical devices. Furthermore, while the applications described above utilize the gellan gum-based films and coatings for fluid impermeability and release rate control, it will be understood that the gellan gum-based films and coating can be used for other applications. It will therefore be appreciated by those skilled in the art that yet other modifications could be made to the provided invention without deviating from its spirit and scope as claimed.

What is claimed is:

1. A method for producing a coating comprising:
a) dissolving a material in a first liquid solution that includes a dissolving liquid, the material including a gellan gum;
b) applying the first liquid solution to a workpiece to form a coating on the workpiece;
c) drying the coating to remove a substantial portion of the dissolving liquid; and
d) exposing the coating to a second liquid solution subsequent to drying, the second liquid solution including a compound that provides ionic cross-linking of the gellan gum of the coating.

2. The method according to claim 1, wherein:
the coating produced by claim 1 is a white color when applied to vascular grafts, the white coating substantially distinguishing the graft from contacting body organs.

3. The method according to claim 2, wherein:
said dissolving liquid comprises water.

4. The method according to claim 2, wherein:
the first liquid solution comprises a polar solvent.

5. The method according to claim 2, wherein:
the compound that provides ionic cross-linking of the gellan gum-based coating comprises a divalent cation.

6. The method according to claim 5, wherein:
the divalent cation comprises Ca²⁺.

7. The method according to claim 6, wherein:
the second liquid solution comprises calcium chloride.

8. The method according to claim 7, wherein:
the second liquid solution comprises a solution of calcium chloride and water with a concentration of calcium chloride by weight in a range between 0.05% and 0.15%.

9. The method according to claim 5, wherein:
the divalent cation is selected from the group consisting of Sr²⁺, Mg²⁺, and Ba²⁺.

10. The method according to claim 2, wherein:
the compound that provides ionic cross-linking of the gellan gum-based coating comprises a multivalent ion.

11. The method according to claim 2, further comprising:
after the drying step and before the exposing step, reapplying the first liquid solution to the workpiece and drying the resultant structure to realize a multi-layer polysaccharide-based coating.

12. The method according to claim 11, further comprising:
controlling the exposing step to provide a gradient of density of ionically cross-linked gellan gum material from an outer portion to an inner portion of the ionically cross-linked gellan gum material.

13. A method of manufacturing an implantable medical device comprising:
a) providing at least one implantable part;
b) dissolving a gellan gum material in a first liquid solution that includes a dissolving liquid;
c) applying the first liquid solution to the at least one implantable part to form a gellan gum-based coating on the at least one implantable part;
d) drying the gellan gum-based coating to remove a substantial portion of the gellan gum-dissolving liquid; and
e) subsequent to drying, exposing the gellan gum-based coating to a second liquid solution, the second liquid solution including a compound that promotes ionic cross-linking of the gellan gum-based coating.

14. The method of claim 13, wherein:
the gellan gum-based coating is a white color that distinguishes the coating from contacting body organs.

15. The method according to claim 14, wherein:
the dissolving liquid comprises water.

16. The method according to claim 14, wherein:
the first liquid solution comprises a polar solvent.

17. The method according to claim 14, wherein:
the compound that provides ionic cross-linking of the gellan gum-based coating comprises a divalent cation.

18. The method according to claim 17, wherein:
the divalent cation comprises Ca²⁺.

19. The method according to claim 18, wherein:
the second liquid solution comprises calcium chloride.

20. The method according to claim 19, wherein:
the second liquid solution comprises a solution of calcium chloride and water with a concentration of calcium chloride by weight in a range between 0.05% and 0.15%.

21. The method according to claim 17, wherein:
the divalent cation comprises at least one multivalent ion selected from the group consisting of Sr²⁺, Mg²⁺, and Ba²⁺.

22. The method according to claim 14, wherein:
the compound that provides ionic cross-linking of the gellan gum-based coating comprises a multivalent ion.

23. The method according to claim 14, further comprising:
after the drying step and before the exposing step, reapplying the first liquid solution to the at least one implantable part and drying the resultant structure to realize a multi-layer gellan gum-based coating.

24. The method according to claim 23, further comprising:
controlling the exposing step to provide a gradient of density of ionically cross-linked gellan gum material from an outer portion to an inner portion of the ionically cross-linked gellan gum material.

25. The method according to claim 14, wherein:
the at least one implantable part comprises a tubular portion of a vascular graft.

26. The method according to claim 25, wherein:
the tubular portion is realized from a woven fabric that is sealed by the gellan gum-based coating such that blood does not leak through its annular wall.

27. The method according to claim 14, wherein:
the at least one implantable part comprises a portion of a stent-graft.

28. The method according to claim 14, wherein:
the at least one implantable part comprises a portion of a stent.

29. A medical device comprising:
at least one film of a material, said at least one film including an ionically cross-linked gellan gum, the film having at least one of the following:
(i) the at least one film being a part of a multi-layer structure comprising a plurality of films, each film including ionically cross-linked gellan gum that are disposed on top of one another;
(ii) the at least one film having a thickness of less than 1 millimeter;
(iii) the at least one film provides a uniform coating; and
(iv) the at least one film is substantially white.

30. The medical device according to claim 29, wherein:
the medical device is selected from the group consisting of stents, stent-grafts, and vascular grafts.
